(19) United States
(12) Patent Application Publication
(10) Pub. No.: US 2008/0242874 A1
(43) Pub. Date: Oct. 2, 2008

(54) Direct Aminolysis

(75) Inventor: Zhengong Bryan Li, East Lyme, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc.

(21) Appl. No.: 12/065,247

(22) PCT Filed: Aug. 21, 2006
(86) PCT No.: PCT/IB06/02337

§ 371 Date: Feb. 28, 2008

Related U.S. Application Data
(60) Provisional application No. 60/713,266, filed on Aug. 31, 2005.

(51) Int. Cl. C07D 205/04

(52) U.S. Cl. 548/952; 548/953

(57) ABSTRACT

In some aspects, the present invention provides a method of preparing a compound of the formula (I) comprising reacting a mesylate compound of the formula (II) by direct aminolysis with a reagent comprising ammonia. The reaction is preferably carried out in a solvent, such as an alcohol, and is preferably carried out in a sealed vessel such as a Parr reactor or the like.

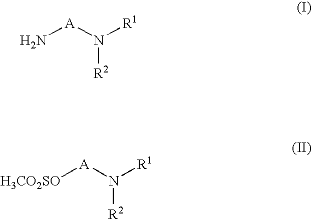

### FIELD AND BACKGROUND

The present invention relates to preparing amines from corresponding mesylates or oximes, including convenient large scale reactions. Others have prepared amines from mesylates and other starting materials. However, there remains a need for improved methods.

### SUMMARY

The section headings used herein are for the reader's convenience and are not limiting to the invention.

In some embodiments, the present invention provides a method of preparing a compound of the formula:

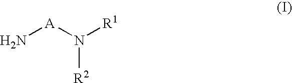

comprising reacting by direct aminolysis a mesylate compound of the formula:

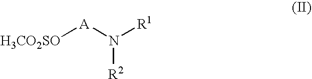

with a reagent comprising ammonia. The reaction is preferably carried out in a solvent, such as an alcohol, and is preferably carried out in a sealed vessel such as a Parr reactor or the like. Without being bound by theory, the sealed vessel advantageously prevents the escape of reagents and can provide relatively high reaction pressures. Advantageously, the invention can be successfully practiced at small or large scale, as desired.

In Formula I, the structure of each of R¹, R², and A in a given synthesis is preferably carried through unchanged from the starting material Formula II. Moreover, unless otherwise indicated, the definitions of R¹, R², and A are the same for all of the generic formulas disclosed herein.

Each R¹ and R² can independently be, e.g., (C₁-C₆)alkyl, (C₂-C₆)alkenyl, or (C₂-C₆)alkynyl, any of which can optionally be substituted by one or more 4 to 6 membered carbocyclic or heterocyclic groups.

A is preferably (C₁-C₆)alkylene, (C₂-C₆)alkenyl, or (C₂-C₆)alkynyl, and R² and A can, together with the nitrogen to which they are attached, form a 4 to 7 membered ring such as azetidinyl, pyrrolidinyl, or piperidinyl.

R¹, R², and A can also be further substituted or can be interrupted by, e.g., oxygen, nitrogen, or sulfur. However, the nature of these substituents is not limiting of the invention. Rather, the aminolysis described herein is generally applicable.

A more detailed non-limiting description follows, including non-limiting examples.

### DETAILED DESCRIPTION

The present invention includes methods of direct aminolysis of mesylates (methane sulfonates) to obtain corresponding amines.

In some embodiments, the present invention provides a method of preparing a compound of the formula:

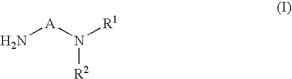

wherein each R¹ and R² is independently (C₁-C₆)alkyl, any of which is optionally substituted by one or more (e.g., 1 to 4) 4 to 6 membered carbocyclic or heterocyclic groups; A is (C₁-C₆)alkylene; and R² and A can, together with the nitrogen to which they are attached, form a 4 to 7 membered ring; comprising reacting a compound of the formula:

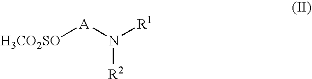

by direct aminolysis with a reagent comprising ammonia, wherein the reaction is carried out in a sealed vessel.

In some embodiments, R² and A, together with the nitrogen to which they are attached, form an azetidinyl ring.

In some embodiments, R¹ is benzhydryl. In some embodiments, R¹ is benzhydryl, and R² and A, together with the nitrogen to which they are attached, form an azetidinyl ring.

In some embodiments, the reaction is carried out in a solvent comprising an alcohol, which can comprise isopropyl alcohol and/or methanol.

In some embodiments, the reagent is added to the vessel as comprising aqueous ammonium hydroxide. In some embodiments, the reagent is added to the vessel as ammonia in an alcohol carrier, such as methanol. For example, 28% aqueous ammonium hydroxide can be used in a reaction with isopropanol as the solvent (e.g., 1 volume ammonium hydroxide to 1.5 volumes alcohol), or 7N ammonia in methanol can be used.

In some embodiments, the yield of Formula I is at least about 70% on a molar basis, or at least about 80% on a molar basis. Moreover, such yields can be obtained at scales affording at least about 500 g or at least about 1000 g of Formula I. According to the invention, the side product Formula I dimer can appear in a ratio to Formula I of about 6:94 or less, or about 4% or less of the resulting products.

In some embodiments, the reaction is heated to at least about 50°, 60°, or at least about 70° C. In some embodiments, the reaction pressure reaches at least about 20, 25, 30, 35, or 40 psi.

In particular, in some embodiments, there is provided a method of preparing a compound of the formula:

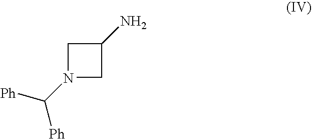

comprising reacting a compound of the formula:

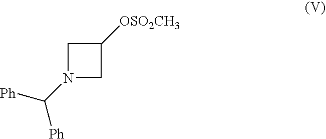

with a reagent comprising ammonia, wherein the reaction is carried out in a solvent comprising isopropanol, in a sealed vessel, wherein the reagent is added to the vessel as aqueous ammonium hydroxide; and wherein Ph is phenyl. In some embodiments, the reaction in the vessel is brought to at least about 50° C. and at least about 25 psi.

In some embodiments, the reaction product is isolated by any suitable combination of evaporation, extraction, or recrystallization (e.g., with or from isopropyl ether).

According to the present invention, there is further provided the preparation of Formula II by reacting a compound of the formula:

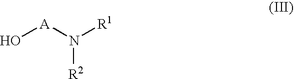

with a mesyl halide (e.g., mesyl chloride) or a mesyl anhydride in a solvent (e.g., acetonitrile). A base such as triethylamine should also be employed. This reaction can be followed by the addition of water, filtration of the product, and use of the Formula II product in the direct aminolysis without prior extraction, purification, or drying. R¹, R², and A in Formula III are as defined for Formula II.

The present invention also provides all of the steps of preparing compounds of Formula I by Swern oxidation of Formula III (e.g., oxalyl chloride, DMSO, −78° C.), condensation (e.g., hydroxylamine hydrochloride), reduction (e.g., LiAlH₄), and isolation (e.g., oxalic acid salt).

### EXAMPLE 1

### Preparation of Formula V

To a 5 L 3-neck round bottom flask was charged 632 g (2.64 mol) of 1-benzhydrylazetidin-3-ol, acetonitrile (1.9 L) and triethylamine (601 g, 1.5 eq.). The mixture was cooled in an ice-acetone bath (−5° C.). Mesyl chloride (436 g, 1.20 eq.) was added by drop funnel while keeping the reaction temperature at <5° C. HPLC showed reaction completion after 15 min. Water (6.3 L) was added, and the reaction mixture was stirred for 2 h at room temperature, and filtered. The filter cake was rinsed with water (2×1 L), and dried under vacuum, and directly subjected to the aminolysis reaction in the next step (Example 2).

### EXAMPLE 2

### Preparation of Formula Iv

The mesylate wet cake (838 g dry weight expected, 2.64 mol) (Example 1) was dissolved in isopropanol at 50° C. The solution was charged to a 2 gallon Parr reactor, followed by the addition of 28 wt % ammonium hydroxide under vacuum (10 vol of 28% NH₄OH and 15 vol of isopropanol). The Parr reactor was sealed, and heated to 71° C. for 3 h (38-40 psi pressure observed). The reaction was assayed by HPLC, and showed reaction completion. The reaction mixture was cooled to room temperature, discharged from the Parr reactor, and concentrated under vacuum. The product was extracted with isopropyl ether (8.4 L). The organic extract was concentrated to ˜4 L under atmospheric pressure, and 159 g (1 eq.) of acetic acid was added, the mixture was stirred for 2 h, and the product (mono acetate salt) was collected by filtration. The solids were dried at 40° C. under vacuum to give 662 g of product (84% yield). About 4% of the Formula IV dimer was observed. ¹H NMR (CD₃OD, 400 MHz) 7.42-7.04 (m, 10H), 4.44 (s, 1H), 3.78-3.62 (m, 1H), 3.43-2.36 (m, 2H), 3.03-2.99 (m, 2H), 1.93 (s, 3H). ¹³C NMR (CD₃OD, 100 MHz) 176.2, 141.4, 128.3, 127.3, 127.2, 77.5, 58.3, 41.2, 22.2.

### EXAMPLE 3

### Preparation of Formula IV

The reaction conditions were similar to Example 2, except that 10 volumes 7N ammonia in methanol was added under vacuum to 15 volumes isopropanol. The reaction was heated to 70-75° C. resulting in a pressure of 40-50 psi. After three hours, the reaction was nearly complete with a ratio of IV to its dimer of 94:6 by HPLC. The product was isolated by evaporation and recrystallized from isopropyl ether to give a 70% yield.

### General Definitions

Unless indicated otherwise in a particular context, each term used herein is to be understood to have its broadest meaning as the term is understood by the ordinarily skilled artisan in the relevant area(s) of art.

Unless otherwise indicated expressly or implicitly herein, the terms “a” or “an” mean at least one. For example, “a compound X” means at least compound X and can include other compounds or materials.

The term “comprising” is open, even where materials are recited in the alternative. For example, “comprising a compound X or Y” means at least compound X or compound Y but can include both compounds X and Y and/or additional compounds and/or components.

The term “alkyl”, as used herein, unless otherwise indicated, means a saturated monovalent hydrocarbon radical including cyclic (“cycloalkyl”), straight and/or branched structure.

The term “alkenyl”, as used herein, unless otherwise indicated, means straight-chain, cyclic, or branched-chain hydrocarbon radicals containing at least one carbon-carbon double bond. Examples of alkenyl radicals include ethenyl, E- and Z-propenyl, isopropenyl, E- and Z-butenyl, E- and Z-isobutenyl, E- and Z-pentenyl, E- and Z-hexenyl, E,E-, E,Z-, Z,E-, Z,Z-hexadienyl, and the like.

The term “alkynyl”, as used herein, unless otherwise indicated, means straight-chain or branched-chain hydrocarbon radicals containing at least one carbon-carbon triple bond. Examples of alkynyl radicals include ethynyl, E- and Z-propynyl, isopropynyl, E- and Z-butynyl, E- and Z-isobutynyl, E- and Z-pentynyl, E, Z-hexynyl, and the like.

The term “aryl”, as used herein, unless otherwise indicated, means a fully aromatic radical containing only carbon atoms in its ring system. Non-limiting examples include phenyl, napthyl, and anthracenyl.

The term “carbocyclic,” as use herein, unless otherwise indicated, means a ring system containing only carbon atoms in the ring system without regard to aromaticity. A carbocyclic moiety can be aryl or non-aryl, wherein non-aryl includes saturated and unsaturated rings, and ring systems having aromatic and/or non-aromatic portions. Examples of carbocyclics include phenyl, naphthyl, cyclohexenyl, and indenyl. The term “4-6 membered carbocyclic” means monocyclic carbocyclic ring systems having 4 to 6 ring carbons.

The term “heteroaryl,” as used herein, unless otherwise indicated, means a fully aromatic radical containing at least one heteroatom in its ring system. Examples of 5-6 membered heteroaryl include, thiophenyl, isoxazolyl, 1,2,3-triazolyl, 1,2,3-oxadiazolyl, 1,2,3-thiadiazolyl, 1,2,4-triazolyl, 1,3,4-oxadiazolyl, 1,3,4-thiadiazolyl, 1,2,5-oxadiazolyl, 1,2,5-thiadiazolyl, pyridyl, pyridazinyl, pyrimidinyl, pyrazinyl, 1,2,4 oxadiazolyl, 1,2,5-triazinyl, 1,3,5-triazinyl, and the like. Heteroaryls include, e.g., 5 and 6 membered monocyclics such as pyrrolyl and pyridinyl. Other examples of heteroaryl include pyridinyl, imidazolyl, pyrimidinyl, pyrazolyl, triazolyl, pyrazinyl, tetrazolyl, furanyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like.

The term “heterocyclic,” as used herein, unless otherwise indicated, means any ring system containing at least one of N, O, or S, and can be heteroaryl or otherwise. Non-aryl heterocyclic groups include saturated and unsaturated systems and can include groups having only 4 atoms in their ring system. The heterocyclic groups include benzo-fused ring systems and ring systems substituted with one or more oxo moieties. Ring sulfur can be in the form of sulfoxide or sulfone where feasible. Included are 4-6 membered ring systems (“4-6 membered heterocyclic”), which include 5-6 membered heteroaryls, and include groups such as azetidinyl and piperidinyl. Heterocyclics can be heteroatom-attached where such is possible. For instance, a group derived from pyrrole can be pyrrol-1-yl (N-attached) or pyrrol-3-yl (C-attached).

What is claimed:

1. A method of preparing a compound of the formula: 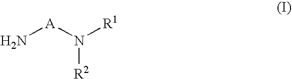
wherein each R¹ and R² is independently (C₁-C₆)alkyl, any of which is optionally substituted by one or more 4-6 membered carbocyclic or heterocyclic groups,
A is (C₁-C₆)alkylene; and
R² and A may, together with the nitrogen to which they are attached; form a 4 to 7 membered ring;
comprising reacting a compound of the formula: 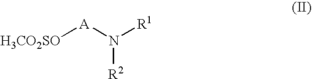
by direct aminolysis with a reagent comprising ammonia, wherein the reaction is carried out in a sealed vessel.

2. The method of claim 1 wherein R² and A, together with the nitrogen to which they are attached, form an azetidinyl ring.

3. (canceled)

4. The method of claim 1, wherein R¹ is benzhydryl and R² and A, together with the nitrogen they are attached, form an azetidinyl ring.

5. The method of claim 4, wherein the reaction is carried out in solvent comprising an alcohol.

6. The method of claim 4, wherein the reaction is carried out in a solvent comprising isopropyl alcohol.

7. The method of claim 4, wherein the reagent is added to the vessel as comprising aqueous ammonium hydroxide.

8. The method of claim 4, wherein the reagent is added to the vessel as comprising ammonia in a carrier comprising an alcohol.

9. The method of claim 4, wherein the yield of Formula I is at least about 70% on a molar basis.

10. The method of claim 4, wherein the yield of Formula I is at least about 80% on a molar basis.

11. The method of claim 10, which yields in one pot at least about 500 g of Formula I.

12. The method of claim 11, wherein the reaction is heated to at least about 50° C.

13. The method of claim 12, wherein the reaction pressure reaches at least about 25 psi.

14. The method of claim 4, further comprising preparing the compound of Formula II by reacting a compound of the formula: 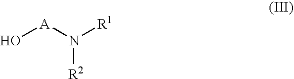
with a mesyl halide or mesyl anhydride in a solvent comprising acetonitrile, followed by the addition of water; filtration of the product, and use of the product Formula II in the direct aminolysis without prior extraction, purification, or drying, wherein R¹, R², and A in Formula III are as defined for Formula II.

15. The method of claim 14, wherein mesyl chloride or mesyl anhydride is used.

16. The method of claim 15, further comprising adding triethylamine to the reaction of Formula III the mesyl halide or mesyl anhydride.

17. A method of preparing a compound of the formula: 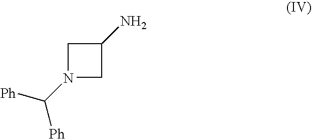
wherein Ph is phenyl,
comprising reacting a compound of the formula: 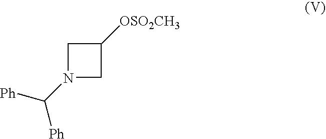
with a reagent comprising ammonia;
wherein the reaction is carried out in a solvent comprising isopropanol, in a sealed vessel; and
wherein the reagent is added to the vessel as aqueous ammonium hydroxide.

18. The method of claim 17, wherein the vessel is brought to at least about 50° C. and at least about 25 psi.

19. A method of preparing a compound of the formula; 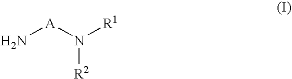
the method comprising reacting, by direct aminolysis, a compound of the formula: 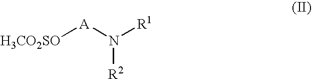
with a reagent comprising ammonia, wherein the reaction is carried out in a sealed vessel;
wherein R¹ is benzhydryl
wherein R² and A, together with the nitrogen to which they are attached, form an azetidinyl ring;
wherein the reagent is added to the vessel as comprising aqueous ammonium hydroxide;
further comprising preparing the compound of Formula II by reacting a compound of the formulas 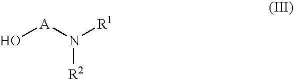
with a mesyl halide or mesyl anhydride in a solvent comprising acetonitrile, followed by, the addition of water filtration of the product, and use of the Formula II product in the direct aminolysis without prior extraction, purification, or drying;
wherein R¹, R², and A in Formula III are the same as in the Formula II product.
